(19) United States
(12) Patent Application Publication
(10) Pub. No.: US 2019/0298442 A1
(43) Pub. Date: Oct. 3, 2019

(54) BI-POLAR TISSUE ABLATION DEVICE AND METHODS OF USE THEREOF

(71) Applicant: RETROVASCULAR, INC., Pleasanton, CA (US)

(72) Inventors: Wayne OGATA, San Ramon, CA (US); Steven MEYER, Oakland, CA (US); Xiang GU, Foster City, CA (US)

(21) Appl. No.: 16/316,568

(22) PCT Filed: Jul. 11, 2017
(86) PCT No.: PCT/US17/41541

§ 371 Date: Jan. 9, 2019

Related U.S. Application Data
(60) Provisional application No. 62/360,911, filed on Jul. 11, 2016.

(51) Int. Cl. A61B 18/14; A61B 18/12; A61B 18/18; A61N 7/02

(52) U.S. Cl. CPC A61B 18/1492; A61B 18/1206; A61B 2018/00267; A61N 7/022; A61B 18/18

(57) ABSTRACT

A tissue ablation device includes a first longitudinal member having a first end configured to be coupled to an energy generator and a second end comprising a first plurality of electrodes that are expandable about a central axis of the first longitudinal member. A second longitudinal member having a first end configured to be coupled to the energy generator and a second end comprising at least one electrode is nested within the first longitudinal member. A method of ablating a tissue using the tissue ablation device is also disclosed.

### FIELD

This application claims priority benefit of U.S. Provisional Patent Application Ser. No. 62/360,911, filed Jul. 11, 2016, which is hereby incorporated by reference in its entirety.

The present invention relates generally to medical methods and devices. In particular, the present invention relates to a bi-polar tissue ablation device and methods of use thereof

### BACKGROUND

RF energy is widely used to coagulate, cut or ablate tissue. In both modalities, monopolar and bipolar, conductive electrodes contact the tissue to be treated. In the monopolar mode, the active electrode is placed in contact with the tissue to be treated and a return electrode with a large surface area is located on the patient at a distance from the active electrode. In the bipolar mode, the active and return electrodes are in close proximity to each other bracketing the tissue to be treated. Sometimes an array of electrodes is used to provide better control over the depth of penetration of the RF field and hence control over the temperatures to which the tissue is heated.

There are a number of disadvantages with each mode. For example, in the monopolar arrangement, because of the large physical separation between the electrodes there are frequent reports of local burning at the electrode sites. For example, this would clearly be undesirable where one of the electrodes will be inside a blood vessel. The other serious issue is the likelihood of forming blood clots. The tissue that is in contact with the electrodes can be coagulated or ablated. In the case of the electrodes being present inside a blood vessel, the formation of dangerous blood clots is undesireable.

In an attempt to overcome the issues described above, various devices and electrode configurations are described in the following patents. U.S. Pat. Nos. 5,366,443 and 5,419,767 describe the use of RF electrodes on a catheter to cross a lesion. These patents describe a bipolar electrode assembly at the distal tip of a catheter that is in contact with the occlusion. The application of RF energy ablates the occlusion and renders the occlusion susceptible for the guidewire to penetrate. This method has the drawback that careful tracking of the occlusion and the ablation process is necessary to avoid trauma to the vessel walls or healthy tissue, since the possibility of short-circuiting of current through healthy tissue instead of the occlusion is high. U.S. Pat. No. 5,419,767 overcomes this limitation to a certain extent through the use of a multiple electrode array. However, this device requires a channel to be pre-created through the occlusion so that the device can be passed through a guidewire traversing this channel.

The present technology is directed to overcoming these and other deficiencies in the art.

### SUMMARY

A tissue ablation device includes a first longitudinal member having a first end configured to be coupled to an energy generator and a second end comprising a first plurality of electrodes that are expandable about a central axis of the first longitudinal member. A second longitudinal member having a first end configured to be coupled to the energy generator and a second end comprising at least one electrode is nested within the first longitudinal member.

A method for ablating a tissue includes advancing a first longitudinal member and a second longitudinal member nested within the first longitudinal member into a tissue region of a body of a patient. The first longitudinal member comprises a first end configured to be coupled to an energy generator and a second end comprising a first plurality of electrodes that are expandable to change the respective positioning between the plurality of electrodes. The second longitudinal member comprises a first end configured to be coupled to the energy generator and a second end comprising at least one electrode. The first plurality of electrodes are expanded to provide a bipolar arrangement between the first plurality of electrodes of the first longitudinal member and the at least one electrode of the second longitudinal member. A delivery of energy to the first and second longitudinal members is initiated from the energy generator to the tissue region of the body to ablate the tissue.

Methods, devices, and systems according to the exemplary embodiments of the present technology provide for bi-polar ablation of tissue. For example, one exemplary application of the present technology is to ablate prostate tissue to relieve the symptoms of conditions such as benign prostatic hyperplasia (BPH) and prostatic carcinoma, where enlargement of the prostate can obstruct the urethra and result in compression and partial or total occlusion of the urethra. As symptoms of prostatic disorders such as BPH often result in obstruction of the urethra, any trans-urethral prostatic treatment methods and devices are likely to be hindered by abnormal tissue occlusion. This is because the device may not be able to properly move within the occluded space to treat the desired area, thus preventing treatment devices from functioning properly or optimally. It is an advantageous aspect of the present technology that it allows resection of tissue, such as prostate tissue, via bi-polar tissue ablation to ablate tissue to weaken, alter, or otherwise treat the tissue instead of, or prior to, surgical resection. Treatments such as tissue ablation could be used to weaken the target tissue by essentially detaching it from the tissue matrix of a body region and thereby allowing easy removal of the treated tissue. More specifically, aspects of the present technology use energy delivered through two longitudinal members for tissue ablation and for recanalization of occluded lumens.

Another exemplary application of the present technology is to ablate a vascular occlusion, such as chronic total occlusions (CTO) or cerebral clots or grafts, such as hemodialysis grafts. Further aspects of the present technology may be employed to ablate other tissues, such as a cardiac structure including, for example, the interatrial septum.

This technology provides a number of advantages including providing safe and effective devices and methods for bi-polar tissue ablation.

### BRIEF DESCRIPTION OF THE DRAWINGS

FIG. 1 is a schematic showing an exemplary tissue ablation system comprising an RF generator connected to a tissue ablation device comprising first and second longitudinal members.

FIG. 2 is a side view of an exemplary longitudinal member that may be employed in the exemplary tissue ablation system illustrated in FIG. 1.

FIGS. 3A-3B are side partial cross-sectional views of exemplary longitudinal members comprising insulators.

FIGS. 3C and 3D show embodiments of an exemplary longitudinal member having a plurality of electrodes configured to expand outwardly in use in an antegrade/retrograde approach in a collapsed state (FIG. 3C) and an expanded state (FIG. 3D).

FIG. 3E is an end cross-sectional view of the exemplary longitudinal member illustrated in FIGS. 3C and 3D with an insulator surrounding the plurality of electrodes.

FIGS. 4A-4B shows an embodiment of longitudinal members where one longitudinal member is nested within another longitudinal member.

FIG. 5A shows an embodiment of longitudinal members where one longitudinal member is nested within another longitudinal member and one of the longitudinal members comprises a plurality of electrodes configured to expand outwardly.

FIG. 5B shows an embodiment of longitudinal members where both members comprise plurality of electrodes configured to expand outwardly.

### DETAILED DESCRIPTION

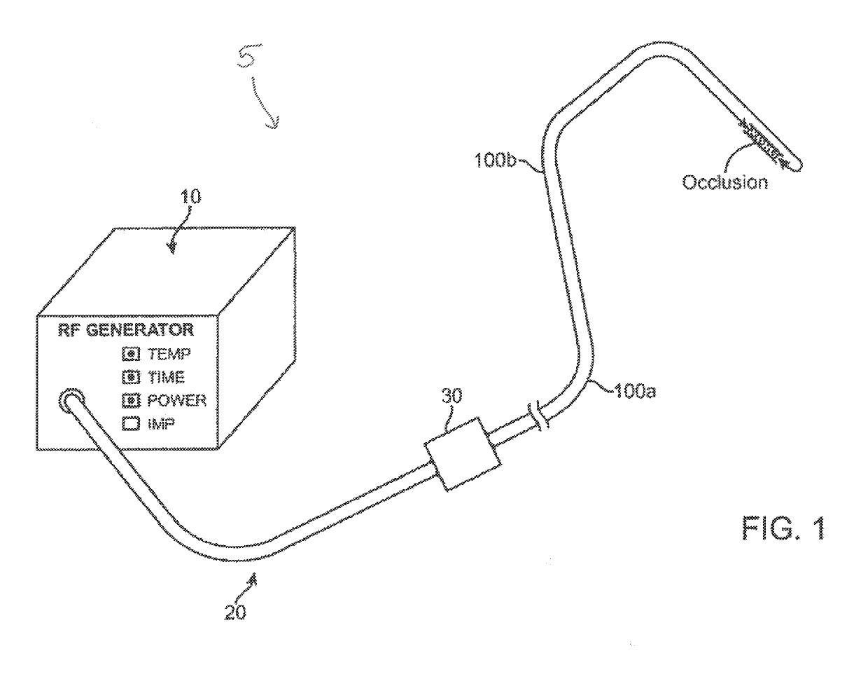

An exemplary tissue ablation system 5 is illustrated in FIG. 1. In this example, the tissue ablation system 5 includes a first longitudinal member 100a and a second longitudinal member 100b coupled to an energy generator 10 through pigtail 20 and connector 30, although the tissue ablation system 5 can include other types and numbers of devices, components, and/or elements in a variety of other configurations. While now shown, the tissue ablation system 5 also may include additional components which are well known to those skilled in the art and thus will not be described here. This technology provides a number of advantages including providing safer and more efficient devices for tissue ablation. More specifically, the present technology provides for bi-polar ablation systems and methods that are advantageous in that they allow for different approaches to a target tissue.

Referring again to FIG. 1, in one exemplary embodiment, the bi-polar tissue ablation system 5 comprises two longitudinal members, the first longitudinal member 100a and the second longitudinal member 100b for delivering energy, such as RF energy, to an occlusion, such as a vascular occlusion, although other types of longitudinal members having other features, such as the additional longitudinal members described below, may be utilized in place of the first longitudinal member 100a and/or the second longitudinal member 100b. Additionally, other numbers of longitudinal members may be utilized in other arrangements. By way of example only, two longitudinal members in a nested arrangement may be employed as described below. Referring back to FIG. 1, in this example, the first longitudinal member 100a and the second longitudinal member 100b are guidewires, but the first longitudinal member 100a and the second longitudinal member 100b may alternatively be any other longitudinal member known in the art such as catheters, micro-catheters, dilating catheters, or combinations thereof.

As indicated in FIG. 1, in this example, the first longitudinal member 100a is configured to approach the target tissue from an antegrade direction and the second longitudinal member 100b is configured to approach the target tissue from a retrograde direction, although other approaches may be utilized to approach the target tissue as described below. The first longitudinal member 100a and the second longitudinal member 100b are configured to have sufficient torsional rigidity and longitudinal flexibility to advance through an occlusion, and to align their electrodes (as described further below) in a direction away from the vessel wall, towards the other longitudinal member, or any combination thereof.

In this example, energy generator 10 (also referred to as a controller) is an RF energy generator that serves as a source of RF energy to be provided to the first longitudinal member 100a and the second longitudinal member 100b. Optionally, in one example the energy generator 10 is a hand-held battery-operated device, although other types of RF generators may be utilized. While the use of RF energy from energy generator 10 for the purpose of ablation is described herein, it should be noted that other energy modalities may be used as well, for example ultrasound energy. In one example, one or both of first longitudinal member 100a and second longitudinal member 100b of the exemplary tissue ablation system 5 of the present technology comprise one or more ultrasound transducers configured to be coupled to the energy generator 10, instead of or in addition to RF electrodes as described below. The ultrasound transducers provide ultrasound energy for ablating an occlusion. In another example, both the first longitudinal member 100a and the second longitudinal member 100b comprise ultrasound transducers and ablate the lesion from an antegrade as well as a retrograde direction. Other energy modalities could include microwave and laser, although additional energy modalities known in the art may be employed.

Referring again to FIG. 1, in this example to provide RF energy from the energy generator 10 to the first longitudinal member 100a and the second longitudinal member 100b, a pigtail 20 connects at its proximal end to the energy generator 10 and terminates at its distal end in a connector 30. The connector 30 is a standard connector that couples input and output signals of the energy generator 10 to the first longitudinal member 100a and the second longitudinal member 100b. An exemplary connector that may be used for the connector 30 would be a locking tool or torque device which can be placed over the first longitudinal member 100a and the second longitudinal member 100b. In such a configuration, the locking tool or torque device is configured to make electrical contact with a portion of the first longitudinal member 100a and the second longitudinal member 100b (such as a corewire of a guidewire) that conducts radiofrequency energy to, or from, one or more electrodes disposed on the first longitudinal member 100a and the second longitudinal member 100b as described below. In such a configuration as described in this example, the locking tool or torque device would also be configured to connect to the energy generator 10, thereby electrically connecting the generator to the first longitudinal member 100a and the second longitudinal member 100b and the electrodes thereon as described below. Exemplary locking connectors that may be utilized as connector 30 may include compressible prongs, screws, sliding rings, or other mechanisms commonly utilized in torque devices.

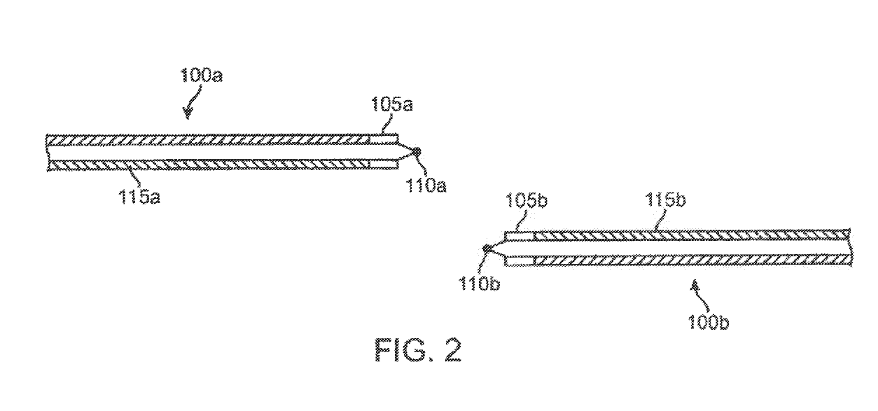

As shown in FIG. 2, in this example, the first longitudinal member 100a and the second longitudinal member 100b have conductive electrodes 105a and 105b, respectively, at their distal ends, although the first longitudinal member 100a and 100b may have other types or numbers of electrodes (and/or other energy delivery elements such as transducers for ultrasound) in other locations. In one example, the electrodes 105a and 105b are located on one side of first longitudinal member 100a and the second longitudinal member 100b, respectively, thereby providing the operating physician with the freedom to allow the electrode-free side of the first longitudinal member 100a and the second longitudinal member 100b to touch the target tissue of the tissue region while still directing the RF energy, or other energy modality, away from the non-targeted tissue. Additionally, this allows the configuration to direct the RF energy away from the non-targeted tissue, such as a vessel wall in the configuration for vascular ablation, thereby minimizing potential RE injury to the vessel wall. In one example, one or both of the first longitudinal member 100a and the second longitudinal member 100b comprises a plurality of electrodes arranged in an array as described in further detail below.

In this example, conductive wires (not shown) connect the electrodes 105a and 105b to the connector 30 to deliver RF energy, by way of example only, from the energy generator 10 to the electrodes 105a and 105b. The exterior of the first longitudinal member 100a and the second longitudinal member 100b are covered by non-conductive layers 115a and 115b, respectively, which sandwich the conductive wires between the first longitudinal member 100a and the second longitudinal member 100b and the non-conductive layers 115a and 115b. In one example, the non-conductive layers 115a and 115b comprise a sheath or a coating. Exemplary materials that may be utilized for the non-conductive layers 115a and 115b may include Teflon, ceramic, polyimide, parylene, or other suitable materials. Exemplary methods that can be employed for coating may include spraying, dipping, vapor deposition, or plasma deposition.

In this example, as further shown in FIG. 2, the first longitudinal member 100a and the second longitudinal member 100b comprise temperature measuring elements 110a and 110b at their distal tips, respectively. The temperature measuring elements 110a and 110b are thermocouples or thermistors that are connected to the connector 30. In another example, pressure measuring elements are placed on the distal ends of the guidewires to detect a change in pressure upon activation of the RF energy, or other energy source.

Referring again to FIG. 1, the energy generator 10 is configured to allow the user to set a maximum temperature, a treatment time period, a level of RF power, or a combination of these control parameters. The treatment time period indicates the period of time over which the RF energy will flow between the electrodes. The maximum temperature setting serves as a threshold temperature for the tissue that is in contact with the electrodes, and the energy generator 10 can be set to reduce or shut off power to one or both electrodes when one or more of the temperature measuring elements 110a and 110b indicate a tissue temperature at or near the threshold.

In one embodiment, the energy generator 10 is capable of measuring the impedance of the tissue between the two electrodes 105a and 105b. Based on the type of the tissue to be ablated, the user can choose the appropriate combination of temperature, treatment time, and the amount of RF energy to be provided to the tissue to achieve a safe and effective treatment. Alternatively, the treatment may proceed with the user manually controlling the parameters during the recanalization procedure, with the user treating the occlusion until recanalization is achieved.

It is noted that energizing an electrode with radiofrequency energy causes the electrode to generate heat. In general, the amount of such heat is proportional to the amount of radiofrequency energy delivered to the electrode, and inversely proportional to the surface area of the electrode. This is because the smaller the surface area of an electrode, the higher the current density passing through that surface area (for a given total current), which in turn causes the electrode to reach correspondingly higher temperatures. In one example, electrodes having sharp points configured to generate a high current density may be employed. By way of example only, ball tip electrodes may be utilized to generate areas of high current density in examples employing radiofrequency energy. In one example, the system is configured to deliver sufficient radiofrequency energy to an electrode such that radiofrequency sparks are generated.

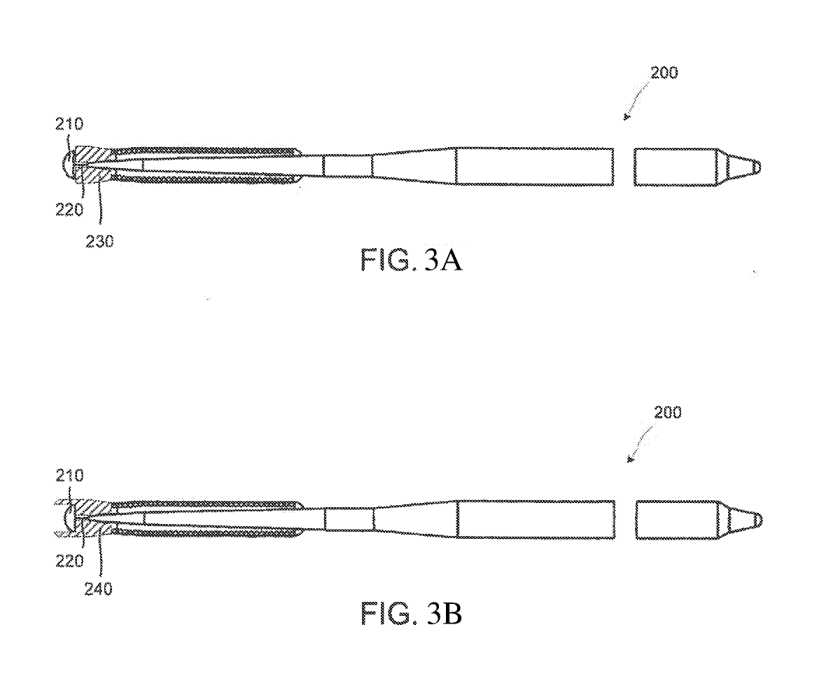

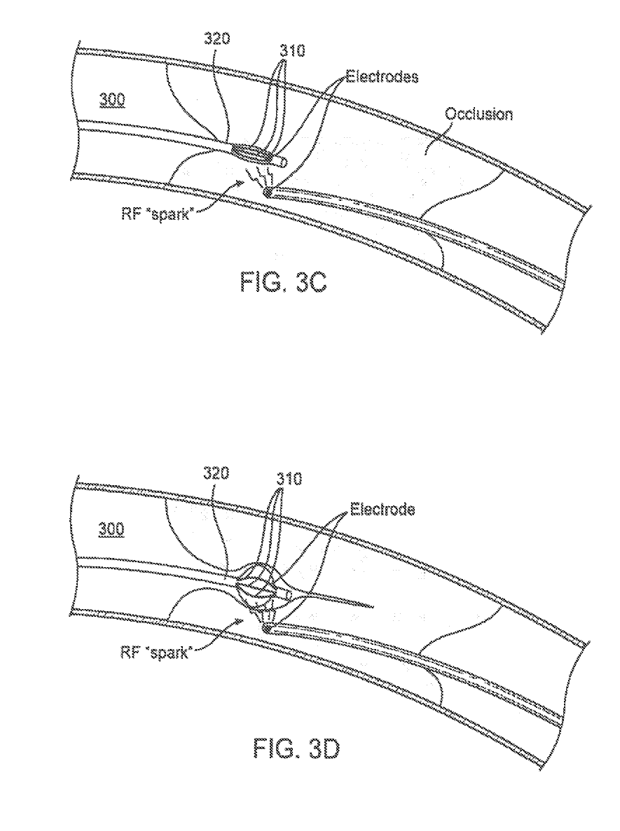

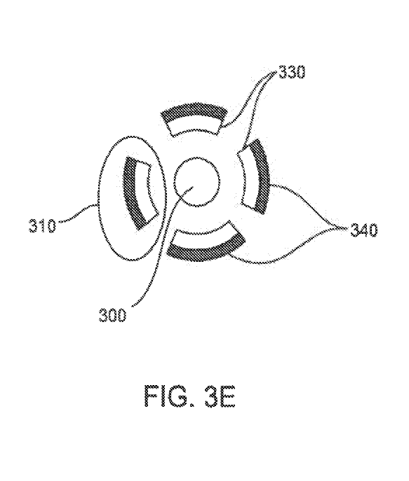

Referring now to FIGS. 3A and 3B, in another example, the conductive wires are insulated by using a heat resistant material, such as an insulator on the longitudinal members, such as guidewires, to protect the device and surrounding tissue from excessive heat. FIG. 3A shows a side partial cross-sectional view of an exemplary longitudinal member 200 comprising an electrode and an insulator, in accordance with an embodiment of the present technology. Longitudinal member 200 is the same in structure and operation as first longitudinal member 100a and/or second longitudinal member 100b except as described below and may be employed in the tissue ablation system 5. The longitudinal member 200 comprises an electrode 210 as its distal tip, although other numbers of electrodes in other locations may be employed. In this example, the longitudinal member 200 is a guidewire with the electrode 210 electrically coupled to the core wire of the longitudinal member 200 via an electrically conductive ribbon 220 or other such electrically conductive connector. An insulator 230 is disposed at a distal portion of the longitudinal member 200 to deflect some of the heat that is generated when the electrode 210 is energized with radiofrequency energy, by way of example only, thereby protecting the rest of the device from such heat. The insulator 230 may wrap around the distal portion of the longitudinal member 200, as shown in FIG. 2A, or it may be configured as a plurality of discrete pieces disposed at the distal portion of the longitudinal member 200. The insulator 230 may or may not directly contact the electrode 210.

In another example, as shown in FIG. 3B, an insulator 240 is configured to protrude forward so that the electrode 210 is recessed. The protruding insulator 240 is configured to extend beyond the electrode 210, thereby recessing the electrode 210. This limits the exposure of the electrode 210 to surrounding tissue, while leaving the electrode 210 sufficiently exposed to create a bipolar arrangement.

While it is possible to have the surface areas of the active and return electrodes be of similar size, in one example an active electrode is configured to have a smaller surface area than a return electrode. This allows the active electrode to generate a current density that is sufficiently high to cause radiofrequency sparks crossing over to the return electrode, while at the same time allowing the return electrode surface area to be sufficiently large so as to maximize its contact with the occlusion and attract sparks from the active electrode. Another advantage of such an embodiment is that the return electrode will likely not reach as high temperatures as the active electrode. In one example, the ratio of the return electrode surface area to the active electrode surface area is configured to be in the range of about 50:1 to 2:1, and preferably about 10:1. In another example, the return electrode is configured in a pigtail design to increase surface area contact with the occlusion.

In another example, such as illustrated in FIG. 3C, a longitudinal member 300 has a plurality of return electrodes 310 configured to expand outwardly in order to spread out and increase surface area contact with the target tissue. The longitudinal member 300 is the same in structure and operation as first longitudinal member 100a and/or second longitudinal member 100b, except as described below. The electrodes 310 are arranged as a plurality of ribs are disposed on a distal end 320 of the longitudinal member 300. Alternatively, separate electrodes may be arranged along a plurality of expandable ribs. In one example, several electrodes are arranged laterally along each of the plurality of ribs. Referring again to FIGS. 3C and 3D, the electrodes 310 are configured to flare out, as shown in FIG. 3D. In a collapsed state, the plurality of electrodes 310 are kept under tension, for example by using a restraining sleeve (not shown), by twisting the plurality of electrodes 310, by exerting a stretching or pulling force on the proximal ends of the plurality of electrodes 310, etc. The longitudinal member 300, with the plurality of electrodes 310 in a collapsed state, may be advanced into the target tissue. Upon releasing the tension or pulling back on the restraining sleeve, the plurality of electrodes 310 flare open. The diameter of the flared plurality of electrodes 310 can be adjusted to conform to the morphology at the site of the targeted tissue, for example, the diameter of a blood vessel. The flaring of the plurality of electrodes 310 allows for a larger volume of tissue to be ablated.

In one example, each of the plurality of electrodes 310, or ribs, comprises an electrode area 330 adjacent to an insulator area 340, as shown in the cross-sectional view of FIG. 3E. In this example, when the plurality of electrodes 310 flare out into a basket-like configuration, the insulator areas 340 are on the outside and the electrode areas 330 are on the inside of the basket-like configuration. Although a basket-like configuration is described, other configurations, such as a helical coil, may be employed for the plurality of electrodes or ribs on which electrodes are located. The configuration shown in FIG. 3E advantageously aids in directing radiofrequency energy inside the basket-like configuration while simultaneously providing protection to the surrounding tissue.

In another example, the electrode areas are confined or concentrated to a portion of the ribs. Confining the electrode areas to a portion of the rib allows for a higher current density at the site of the electrode to more effectively ablate the targeted tissue. In one example, one or more ribs that comprise one or more ball tip electrodes on the inside of the ribs are utilized. In this example, the one or more ball tips are configured as points where energy is transmitted. Alternatively, it is contemplated that in other examples the placement of the electrode areas 330 and insulator areas 340 may be varied. In an optional example, a capture device may be configured to comprise one or more electrode areas for use as return electrodes. Examples of capture devices are disclosed in U.S. Pat. No. 9,119,651, the disclosure of which is hereby incorporated by reference herein in its entirety. In yet an additional optional example, a capture device comprising an aspiration catheter is incorporated to aspirate any residual debris from the ablation. Furthermore, the basket-like configuration, especially when expanded may serve as a stabilization element by anchoring the longitudinal member within the tissue region.

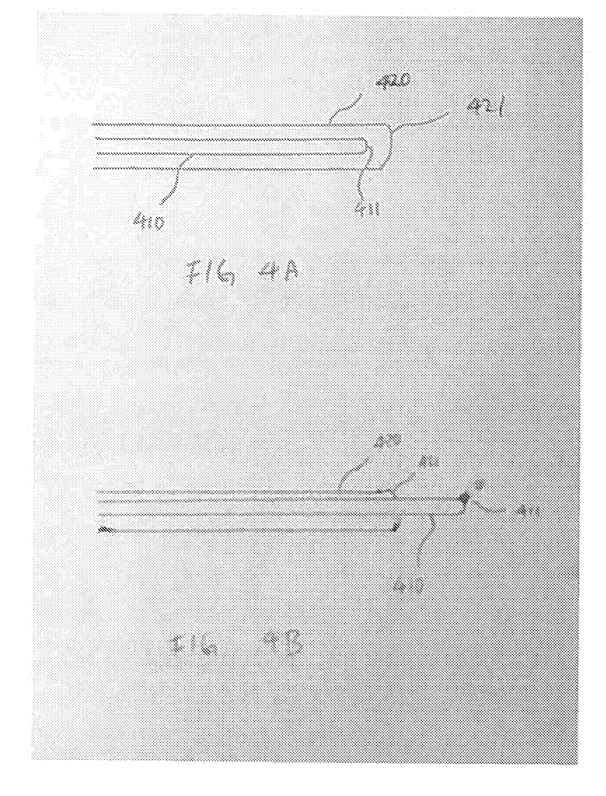

In another example, as seen in FIG. 4A, a first longitudinal member 410 is nested within the second longitudinal member 420 prior to deployment, wherein the first longitudinal member 410 is the nested longitudinal member and the second longitudinal member 420 is configured to be the host longitudinal member. Although the first longitudinal member 410 is described as being nested in the second longitudinal member 420, the first and second designations are for description only and either longitudinal member may be nested in the other in this example. The longitudinal member 410 and 420 are the same in structure and operation as first longitudinal member 100a and/or second longitudinal member 100b, except as described below, and may be employed in the tissue ablation system 5 as shown in FIG. 1.

The longitudinal members 410 and 420 comprise conductive electrodes 411 and 421, respectively, located at their distal ends, although the longitudinal members 410 and 420 may have other types and numbers of electrodes in other locations. In this example, the two longitudinal members 410 and 420 may be advanced into the tissue region in tandem. Once the two longitudinal members 410 and 420 are near the target tissue, as seen in FIG. 4B, the first longitudinal member 410 is extended from the second longitudinal member 420 such that the first longitudinal member 410 is distal to the second longitudinal member 420. After the first longitudinal member 410 assumes the extended configuration, the two longitudinal members 410 and 420, through electrodes 411 and 421, form a bi-polar configuration such that a portion of the target tissue between or near the electrodes 411 and 421 of the first and the second longitudinal members 410 and 420 are subject to ablation when energy is delivered between the two longitudinal members 410 and 420 via the electrodes 411 and 421.

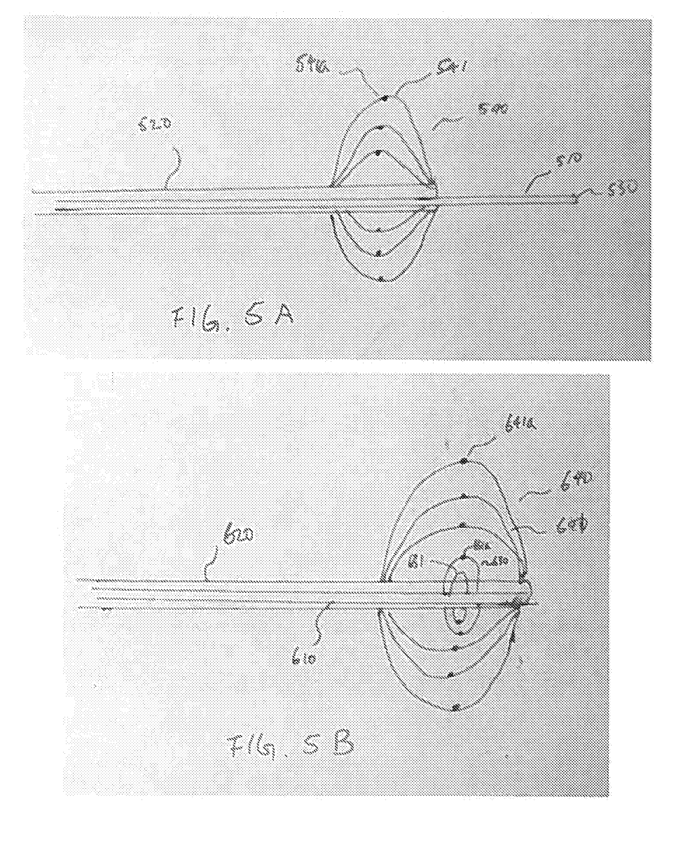

In another example, as seen in FIG. 5A, first and/or second longitudinal members 510 and 520 are configured with a plurality of electrodes 540 configured to expand outwardly in a basket-like configuration in order to spread out and increase surface area contact with the target tissue, although other configurations such as a helical coil may be utilized. The longitudinal members 510 and 520 are the same in structure and operation as first longitudinal member 100a and/or second longitudinal member 100b, except as described below.

In this example, contemporaneously or sequentially with the deployment of the first longitudinal member 510, the plurality of electrodes 540 configured as ribs 541 of the second longitudinal member 520 are configured to transform from a collapsed state to the expanded state as described above. In one example, the one or more ribs comprises one or more ball tips 541a on the inside of the ribs that serve as the electrodes. In one example, the ball tips 541a are configured as points where energy is transmitted with a high current density, although other electrode configurations that provide sharp points to create high current densities may be employed. Thereafter, the two longitudinal members 510 and 520 form a bi-polar configuration such that a portion of the target tissue between or near the first and the second longitudinal members 510 and 520 is subject to ablation when energy is delivered between the two longitudinal members 510 and 520 via the plurality of electrodes 540 disposed on the second longitudinal member 520 and the electrode 530 on the first longitudinal member 510. In another example, the two longitudinal members 510 and 520 are separately advanced to the target tissue.

In one aspect of the nested examples illustrated in FIGS. 4A-5B, the systems and methods are configured for prostate ablation. In this example, the nested longitudinal members are inserted into a lumen of the urethra within the prostate. Thereafter, once the longitudinal members are at or near the target tissue, such as tissue for treatment of BPH, the nested longitudinal member is deployed within or near the host longitudinal member. Thereafter, the plurality of electrodes are expanded to the basket configuration, although other configurations such as a helical coil may be utilized. When expanded, the basket-like configuration serves as a stabilization element by anchoring the longitudinal members within the urethra. Thereafter, energy is delivered between the host and the nest longitudinal members to achieve bi-polar tissue ablation.

In another example, as seen in FIG. 5B, both first and the second longitudinal members 610 and 620 may be configured with a plurality of electrodes 630 and 640 configured to expand outwardly in a basket-like configuration in the expanded configuration. The longitudinal members 610 and 620 are the same in structure and operation as first longitudinal member 100a and/or second longitudinal member 100b, except as described below. In one example, the basket-like configuration of the plurality of electrodes 630 of the first longitudinal member 610 is configured to be nested within the basket-like configuration of the plurality of electrodes 640 of the second longitudinal member 620. In this configuration, the basket-like configuration of the plurality of electrodes 640 of the second longitudinal member 620 is bigger to host the basket-like configuration of the plurality of electrodes 630 of the first longitudinal member 610, even in the expanded configuration. In one example, the basket-like configurations of the plurality of electrodes 630 and 640 may be deployed simultaneously or sequentially. In another embodiment, the diameter in the expanded configuration of both longitudinal members 610 and 620 are the same. In one example, the longitudinal members 610 and 620 have one or more ribs 631 and 641 that comprise one or more ball tips 641a and 631a on the inside of the ribs. In this example, the ball tips 641a and 631a are configured as points where energy is transmitted in a high current density although other high current density electrode configurations may be utilized.

Thereafter, the two longitudinal members 610 and 620 form the bi-polar configuration such that a portion of the target tissue between or near the first and the second longitudinal members 610 and 620 is subject to ablation when energy is delivered between the two longitudinal members 610 and 620 via the plurality of electrodes 630 and 640.

In yet another example, a single longitudinal member comprises two sets of the plurality of electrodes configured to expand outwardly in a basket-like configuration in the expanded configuration. The first basket of the plurality of electrodes is configured to be nested within the second basket of the plurality of electrodes. In this configuration, the two baskets of electrodes form the bi-polar configuration such that a portion of the target tissue between the two baskets are subject to ablation when energy is delivered between the two baskets of electrodes.

While the present embodiments have been described primarily with reference to transurethral treatment of the prostate, it is contemplated that certain aspects of the embodiments may also be used to treat and modify other organs such as brain, heart, lungs, intestines, eyes, skin, kidney, liver, pancreas, stomach, uterus, ovaries, testicles, bladder, ear, nose, etc., soft tissues such as bone marrow, adipose tissue, muscle, glandular tissue, spinal tissue, etc., hard biological tissues such as teeth, bone, etc., as well as body lumens and passages such as the sinuses, ureter, colon, esophagus, lung passages, blood vessels, etc. The devices disclosed herein may be inserted through an existing body lumen, or inserted through solid body tissue.

Another aspect of the present invention relates to the delivery of energy in the bipolar arrangement. In particular, the energy can be delivered sequentially to each individual rib in an array so as to concentrate energy deliver between a first electrode on the nested longitudinal member and a second electrode located on an individual rib. Energy is delivered sequentially to each individual rib in a circular fashion so as to scan 360 degrees within the targeted tissue. Alternatively, the energy can be delivered contemporaneously.

An exemplary operation of the tissue ablation system 5 to perform an exemplary method for ablating a tissue will now be described with reference to FIGS. 1 and 5A.

First the longitudinal members 510 and 520 are delivered into a tissue region of a body of a patient, such as prostrate tissue or a vascular occlusion by way of example only, with the longitudinal member 510 nested within the longitudinal member 520. The longitudinal members 510 and 520 are coupled to the energy generator 10, such as a radiofrequency energy generator. The longitudinal member 510 comprises a single electrode 530 on its distal end, while the longitudinal member 520 includes a plurality of expandable electrodes 540 on its distal end.

Next, the plurality of electrodes 540 on the longitudinal member 520 are expanded to provide a bipolar arrangement between the plurality of electrodes 540 of the longitudinal member 520 and the electrode 530 of the longitudinal member 510. By way of example only, the plurality of electrodes 540 may be expanded into a basket-like configuration, although other expanded configurations, such as a helical coil may be utilized. In another example, a plurality of ribs 541 with electrodes configured to generate a high current density such as ball-tip electrodes 541a may be employed for the basket-like configuration. In one example, either concurrently or sequentially with expanding the plurality of electrodes 540, the longitudinal member 510 may be extended from its nested position in the longitudinal member 520 to alter the distance between the electrode 530 and the plurality of electrodes 540 prior to tissue ablation.

A delivery of energy to the longitudinal members 510 and 520 from the energy generator 10 to the tissue region of the body is then initiated to ablate the tissue. In one example, the energy is radiofrequency energy, although other energy modalities may be used. The position of the of the longitudinal member 510 from its nested position in longitudinal member 520 may be adjusted to change the area of tissue ablated by the delivery of energy.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A tissue ablation device comprising:
a first longitudinal member having a first end configured to be coupled to an energy generator and a second end comprising a first plurality of electrodes that are expandable about a central axis of the first longitudinal member; and
a second longitudinal member having a first end configured to be coupled to the energy generator and a second end comprising at least one electrode, wherein the second longitudinal member is nested within the first longitudinal member.

2. The device of claim 1, wherein the first plurality of electrodes of the first longitudinal member are expandable in a basket-like configuration.

3. The apparatus of claim 2, wherein the first plurality of electrodes are located laterally along the basket-like configuration.

4. The device of claim 1, wherein the first plurality of electrodes of the first longitudinal member are expandable in a helical coil.

5. The device of claim 1 further comprising:
an insulator located on at least a portion of the plurality of first electrodes of the first longitudinal member that is distal to the central axis of the first longitudinal member.

6. The device of claim 1, wherein the second longitudinal member comprises a second plurality of electrodes that are expandable about a central axis of the second longitudinal member.

7. The device of claim 6, wherein the second plurality of electrodes are located within the first plurality of electrodes when both the first plurality of electrodes and the second plurality of electrodes are in an expanded state.

8. The device of claim 6, wherein the second plurality of electrodes of the second longitudinal member are expandable in a basket-like configuration.

9. The device of claim 6, wherein the second plurality of electrodes of the second longitudinal member are expandable in a helical coil.

10. The device of claim 1, wherein the second longitudinal member is extendable from the first longitudinal member to change a distance between the at least one electrode on the second longitudinal member and the first plurality of electrodes on the first longitudinal member.

11. The device of claim 1, wherein the first plurality of electrodes or the at least one electrode are configured to provide an area of high current density.

12. The device of claim 11, wherein the first plurality of electrodes or the at least one electrode are ball-tip electrodes located on first plurality of expandable ribs.

13. A method for ablating a tissue, the method comprising:
advancing a first longitudinal member and a second longitudinal member nested within the first longitudinal member into a tissue region of a body of a patient, wherein the first longitudinal member comprises a first end configured to be coupled to an energy generator and a second end comprising a first plurality of electrodes that are expandable to change the respective positioning between the plurality of electrodes and the second longitudinal member comprises a first end configured to be coupled to the energy generator and a second end comprising at least one electrode;
expanding the first plurality of electrodes to provide a bipolar arrangement between the first plurality of electrodes of the first longitudinal member and the at least one electrode of the second longitudinal member; and
initiating a delivery of energy to the first and second longitudinal members from the energy generator to the tissue region of the body to ablate the tissue.

14. The method of claim 13, wherein the tissue region comprises prostate tissue or a vascular occlusion.

15. The method of claim 13, wherein the first plurality of electrodes of the first longitudinal member are expandable in a basket-like configuration.

16. The method of claim 15, wherein the first plurality of electrodes are located laterally along the basket-like configuration.

17. The method of claim 13, wherein the first plurality of electrodes of the first longitudinal member are expandable in a helical coil.

18. The method of claim 13, wherein the second longitudinal member comprises a second plurality of electrodes that are expandable about a central axis of the second longitudinal member, the method further comprising:
expanding the second plurality of electrodes to provide the bipolar arrangement between the first plurality of electrodes of the first longitudinal member and the second plurality of electrodes of the second longitudinal member.

19. The method of claim 13, wherein the second longitudinal member is extendable from the first longitudinal member to change a distance between the at least one electrode on the second longitudinal member and the first plurality of electrodes on the first longitudinal member, the method further comprising:
extending the second longitudinal member from the first longitudinal member prior to initiating the energy delivery.

20. The method of claim 13 further comprising:
adjusting the position of the of the longitudinal member to ablate a second tissue region.
